(19) United States
(12) Patent Application Publication
(10) Pub. No.: US 2026/0285849 A1
(43) Pub. Date: Sep. 24, 2026

(54) METHYLOPHIOPOGONONE A DERIVATIVE, AND PREPARATION AND USE THEREOF

(71) Applicant: INSTITUTE OF MATERIA MEDICA, CHINESE ACADEMY OF MEDICAL SCIENCES, Beijing (CN)

(72) Inventors: Jinlan ZHANG, Beijing (CN); Hua SUN, Beijing (CN); Dongmei WANG, Beijing (CN); Yanxin LIU, Beijing (CN); Zihan CHEN, Beijing (CN); Zhiwei CHEN, Beijing (CN); Ning SHENG, Beijing (CN); Zhe WANG, Beijing (CN); Bowen ZHENG, Beijing (CN); Modi LIN, Beijing (CN)

(21) Appl. No.: 19/144,675

(22) PCT Filed: Jul. 23, 2023
(86) PCT No.: PCT/CN2023/108779

§ 371 Date: Jun. 9, 2026

(30) Foreign Application Priority Data
Jul. 31, 2022 (CN) 202210919793.1

(51) Int. Cl. C07D 407/06; A61K 31/36; A61P 9/00; A61P 9/10; A61P 9/12

(52) U.S. Cl. CPC C07D 407/06; A61K 31/36; A61P 9/00; A61P 9/10; A61P 9/12

(57) ABSTRACT

Provided are a methylophiopogonone A derivative, and the preparation and the use thereof. The present invention specifically relates to three derivatives of methylophiopogonone A, which derivatives have the chemical structures of formula I, formula II and formula III; and a method for preparing the derivatives, and the use of the derivatives in the preparation of a drug for preventing and/or treating cardiovascular diseases, etc.

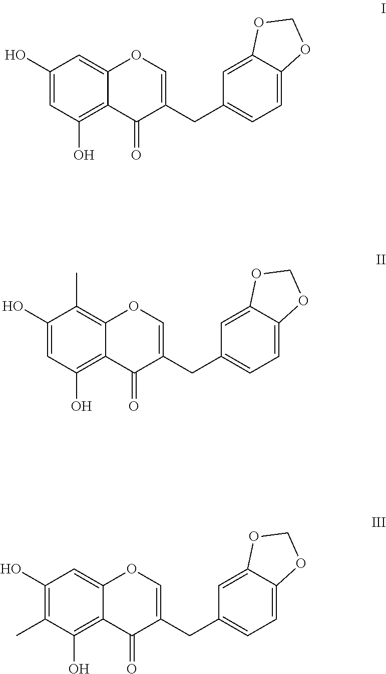 [SMILES: O=c1c(Cc2ccc3c(c2)OCO3)coc2cc(O)cc(O)c12 ; Cc1c(O)cc(O)c2c(=O)c(Cc3ccc4c(c3)OCO4)coc12 ; Cc1c(O)cc2occ(Cc3ccc4c(c3)OCO4)c(=O)c2c1O]

### TECHNICAL FIELD

This invention belongs to the field of medicinal chemistry, specifically involving three types of methyl ophiopogonone A class derivatives (formulas I, II, and III), their preparation, and their application, especially in the treatment and/or prevention of cardiovascular diseases and related diseases.

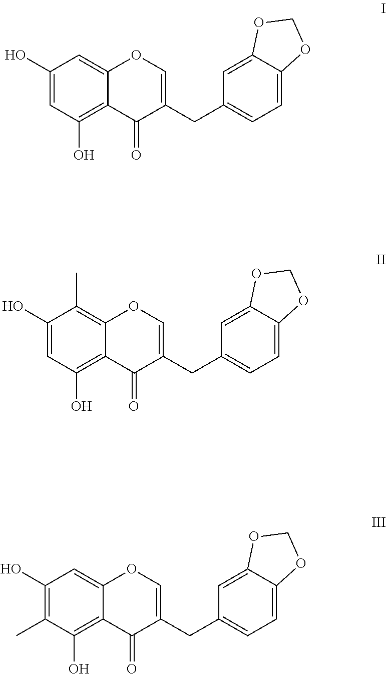 [SMILES: O=c1c(Cc2ccc3c(c2)OCO3)coc2cc(O)cc(O)c12 ; Cc1c(O)cc(O)c2c(=O)c(Cc3ccc4c(c3)OCO4)coc12 ; Cc1c(O)cc2occ(Cc3ccc4c(c3)OCO4)c(=O)c2c1O]

### BACKGROUND TECHNOLOGY

Cardiovascular diseases (CVD) are the leading cause of death globally, accounting for 31.5% of all deaths worldwide and 45.0% of deaths due to non-communicable diseases, higher than those caused by tumors and other diseases, with a continuously increasing prevalence. According to the “China Cardiovascular Health and Disease Report 2021,” it is estimated that there are 330 million people with cardiovascular diseases in China, representing about 23% of the total population, including 11.39 million with coronary heart disease, 8.9 million with heart failure, 245 million with hypertension, 5 million with pulmonary heart disease, 4.87 million with atrial fibrillation, and 2.5 million with rheumatic heart disease. The age of onset is also gradually becoming younger. The total hospitalization costs for cardiovascular and cerebrovascular diseases are increasing year by year, with an average growth rate far exceeding the growth rate of the Gross National Product.

In the nearly 40 years, there has been significant progress in the treatment of cardiovascular diseases, including beta-blockers, calcium channel blockers, diuretics, renin-angiotensin-aldosterone system drugs, lipid-regulating drugs, vasodilators, sinoatrial node channel blockers, drugs for improving myocardial metabolism, and traditional Chinese medicine preparations such as Danshen tablets, Su Xin Jiu Xin pills, and Ginkgo biloba preparations. However, in recent years, the development of new drugs in this field has encountered a bottleneck. Except for the repurposing of old drugs (such as the sodium-glucose co-transporter 2 inhibitors empagliflozin and dapagliflozin) and a few new target candidate drugs (Vericiguat and Omecamtiv Mecarbil), the overall development has significantly lagged behind that of cancer drugs and autoimmune disease drugs. Existing drugs cannot meet the needs of clinical treatment, and there is an urgent need to develop new drug therapies. At the same time, the current standard treatment still carries cardiovascular risks. Patients receiving secondary prevention still have a considerable residual cardiovascular risk, with a risk of major adverse cardiovascular events remaining at 4-12%.

Discovering and developing innovative drugs based on traditional Chinese medicine and natural products has always been an important pathway for new drug creation. Chinese original and internationally recognized innovative drugs, such as artemisinin, dihydroartemisinin, huperzine A, binazole, and butylphthalide, all originate from traditional Chinese medicine. Methyl ophiopogonone A is a component contained in the traditional Chinese medicine Ophiopogon japonicus. Studies have shown that it has anti-non-alcoholic fatty liver and hepatoprotective activities. Our research has found that it has cardiovascular disease prevention and treatment activities. In this invention, we have used methyl ophiopogonone A as a lead compound and discovered a class of methyl ophiopogonone A derivatives with more optimal activity, and we have conducted pharmacological evaluations.

### Content of the Invention

The technical problem addressed by this invention is to provide three derivatives of methyl ophiopogonone A class, their preparation methods, and their application in the preparation of drugs for the prevention and/or treatment of cardiovascular-related diseases.

To address the technical problem of this invention, the invention provides the following technical solutions:

The first aspect of the technical solution of this invention is to provide three types of methyl ophiopogonone A class derivatives with formulas I, II, and III, as well as their pharmaceutically acceptable salts, which have the following structural formulas:

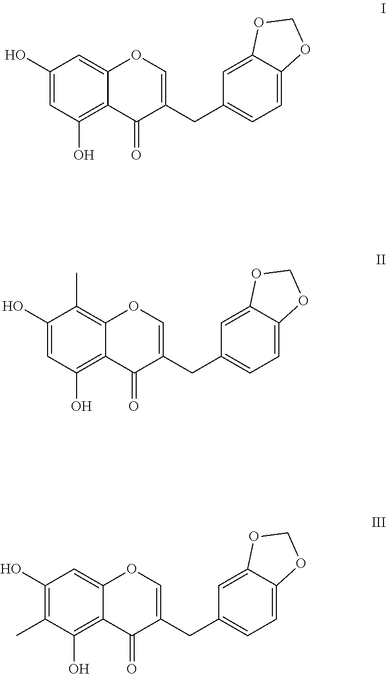 [SMILES: O=c1c(Cc2ccc3c(c2)OCO3)coc2cc(O)cc(O)c12 ; Cc1c(O)cc(O)c2c(=O)c(Cc3ccc4c(c3)OCO4)coc12 ; Cc1c(O)cc2occ(Cc3ccc4c(c3)OCO4)c(=O)c2c1O]

The second aspect of the technical solution of the present invention is to provide a preparation method for the three types of methyl ophiopogonone A class derivatives with formulas I, II, and III, as well as their pharmaceutically acceptable salts, as described in the first aspect.

The methyl ophiopogonone A class derivative I of the present invention can be obtained by the following method, which comprises the steps of:

Under basic conditions, reacting 2, 4, 6-trihydroxyacetophenone (1) with a halomethoxymethyl ether to produce a compound (2); reacting the compound (2) with piperonal under basic conditions to produce a compound (3); reducing the double bond of the compound (3) to produce a compound (4); reacting the compound (4) with phosphorus oxychloride and N, N-dimethylformamide to produce a compound I.

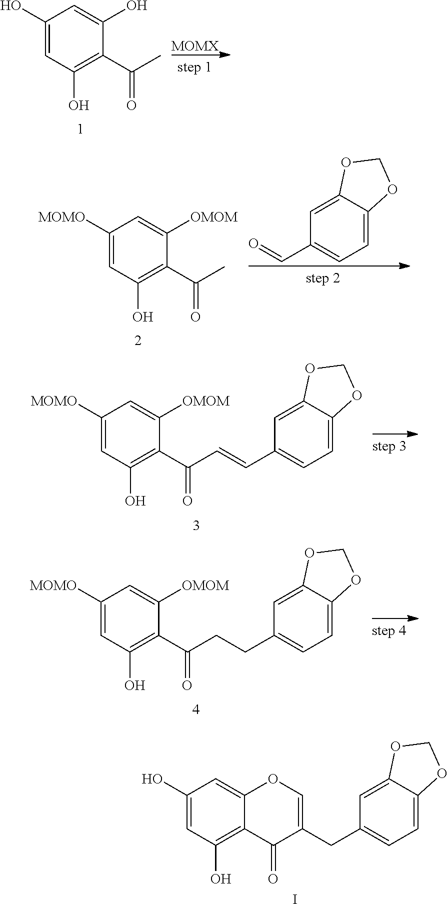

Preferably, in the first step, the base is triethylamine or N, N-diisopropylethylamine, and the halomethoxymethyl ether is bromomethoxymethyl ether. In the second step, the base is sodium hydride or sodium alkoxide, such as KOH/EtOH. In the third step, the reducing agent is palladium on carbon/hydrogen or stannous chloride.

More preferably, in the first step, the base is N, N-diisopropylethylamine. In the second step, the base is sodium hydride, and the reaction is conducted in the presence of a solvent such as tetrahydrofuran or N, N-dimethylformamide. In the third step, the reducing agent is palladium on carbon/hydrogen.

The methyl ophiopogonone A class derivatives II and III of present invention can be obtained by a method comprising the following steps:

reducing 2, 4, 6-trihydroxybenzaldehyde to 2, 4, 6-trihydroxytoluene, then reacting with acetic anhydride to produce a compound (5); the compound (5) with a halomethoxymethyl ether under basic conditions to produce a compound (6); the compound (6) i with piperonal under basic conditions to produce a compound (7); reducing the double bond of the compound (7) to produce a compound (8); reacting the compound (8) with phosphorus oxychloride and N, N-dimethylformamide to produce compounds II and III.

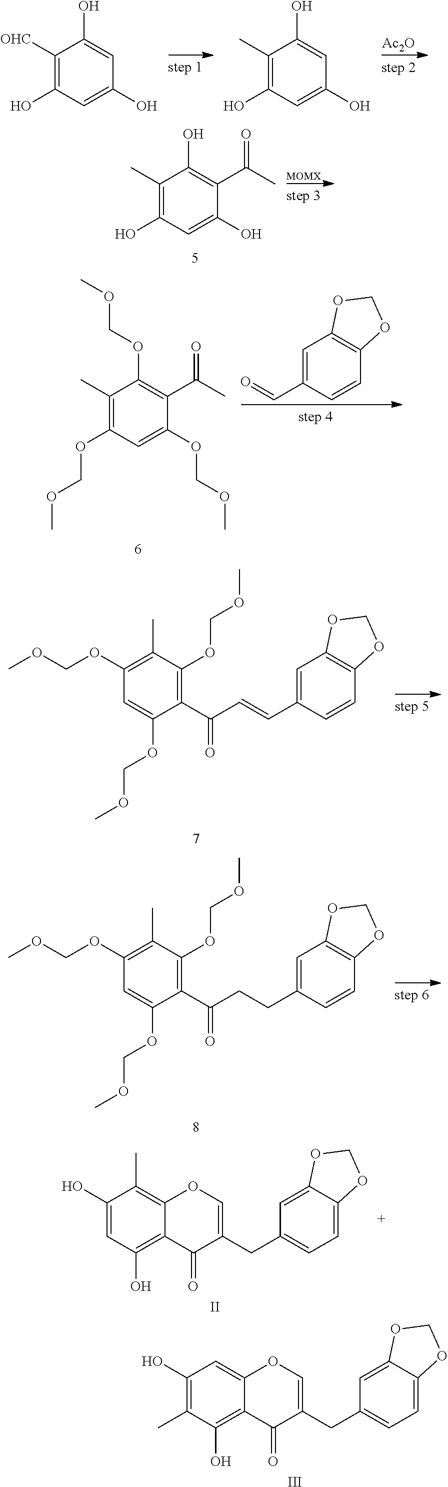

Preferably, in the first step, the reducing agent is a metallic reducing agent or a metal borohydride. In the third step, the base is triethylamine or N, N-diisopropylethylamine. In the fourth step, the base is sodium hydride or sodium alkoxide, such as KOH/EtOH. In the fifth step, the reducing agent is palladium on carbon/hydrogen or stannous chloride.

More preferably, in the first step, the reducing agent is a metallic reducing agent. In the third step, the base is N, N-diisopropylethylamine. In the fourth step, the base is sodium hydride, and the reaction is conducted in the presence of tetrahydrofuran. In the fifth step, the reducing agent is palladium on carbon/hydrogen.

The third aspect of the technical solution of this invention provides a pharmaceutical composition, which comprises an effective amount of the methyl ophiopogonone A class derivatives and their pharmaceutically acceptable salts as described in the first aspect of this invention, for the treatment and/or prevention of diseases, as well as one or more optional pharmaceutically acceptable carriers or excipients.

The pharmaceutical composition can be prepared according to methods well-known in the art. It can be formulated into any dosage form suitable for human or veterinary use by combining the compounds of this invention with one or more pharmaceutically acceptable solid or liquid excipients and/or adjuvants. The content of the compounds of this invention in their pharmaceutical compositions is usually in the range of 0.1-95% by weight.

The compounds of this invention or their pharmaceutical compositions can be administered in unit dosage forms, and the routes of administration can be enteral or parenteral, such as oral, intravenous, intramuscular, subcutaneous, nasal, oral mucosa, ocular, pulmonary and respiratory tract, dermal, vaginal, rectal, etc.

The dosage forms for administration can be liquid, solid, or semi-solid. Liquid dosage forms can include solutions (including true solutions and colloidal solutions), emulsions (including o/w, w/o, and multiple emulsions), suspensions, injections (including aqueous injections, powder injections, and infusions), eye drops, nasal drops, washes, and liniments, etc.; solid dosage forms can include tablets (including ordinary tablets, enteric-coated tablets, buccal tablets, dispersible tablets, chewable tablets, effervescent tablets, orally disintegrating tablets), capsules (including hard capsules, soft capsules, enteric-coated capsules), granules, powders, microspheres, drop pills, suppositories, films, patches, aerosols (powder aerosols), sprays, etc.; semi-solid dosage forms can include ointments, gels, pastes, etc.

The compounds of this invention can be formulated into conventional dosage forms, as well as into sustained-release, controlled-release, targeted formulations, and various particulate drug delivery systems.

To formulate the compounds of this invention into tablets, a wide range of excipients known in the field can be used, including diluents, binders, wetting agents, disintegrants, lubricants, and solubilizers. Diluents can be starch, dextrin, sucrose, glucose, lactose, mannitol, sorbitol, xylitol, microcrystalline cellulose, calcium sulfate, calcium hydrogen phosphate, calcium carbonate, etc.; wetting agents can be water, ethanol, isopropanol, etc.; binders can be starch paste, dextrin, syrup, honey, glucose solution, microcrystalline cellulose, acacia mucilage, gelatin solution, sodium carboxymethyl cellulose, methyl cellulose, hydroxypropyl methyl cellulose, ethyl cellulose, acrylic resin, carbomer, polyvinylpyrrolidone, polyethylene glycol, etc.; disintegrants can be dry starch, microcrystalline cellulose, low-substituted hydroxypropyl cellulose, cross-linked polyvinylpyrrolidone, cross-linked sodium carboxymethyl cellulose, sodium carboxymethyl starch, sodium bicarbonate with citric acid, polyoxyethylene sorbitan fatty acid esters, sodium lauryl sulfate, etc.; lubricants and solubilizers can be talc, silicon dioxide, stearates, tartaric acid, liquid paraffin, polyethylene glycol, etc.

Tablets can also be further formulated into coated tablets, such as sugar-coated tablets, film-coated tablets, enteric-coated tablets, or bilayer and multilayer tablets.

To formulate the dosage unit into capsules, the active ingredient, the compounds of this invention, can be mixed with diluents and solubilizers, and the mixture can be directly placed into hard or soft capsules. Alternatively, the active ingredient, the compounds of this invention, can first be formulated into granules or microspheres with diluents, binders, and disintegrants, and then placed into hard or soft capsules. The various diluents, binders, wetting agents, disintegrants, and solubilizers used for the preparation of tablets of the compounds of this invention can also be used for the preparation of capsules of the compounds of this invention.

To formulate the compounds of this invention into injections, solvents such as water, ethanol, isopropanol, propylene glycol, or their mixtures can be used, with the addition of appropriate amounts of solubilizers, co-solvents, pH adjusters, and osmotic pressure regulators commonly used in the field. Solubilizers or co-solvents can be poloxamers, lecithin, hydroxypropyl-β-cyclodextrin, etc.; pH adjusters can be phosphates, acetates, hydrochloric acid, sodium hydroxide, etc.; osmotic pressure regulators can be sodium chloride, mannitol, glucose, phosphates, acetates, etc. If preparing lyophilized powder for injection, mannitol, glucose, etc., can also be added as fillers.

Additionally, if necessary, coloring agents, preservatives, flavorings, sweeteners, or other additives can be added to the pharmaceutical preparations.

To achieve the therapeutic purpose and enhance the therapeutic effect, the drugs or pharmaceutical compositions of this invention can be administered by any known method of administration.

The dosage of the pharmaceutical composition of the compounds of this invention can vary widely depending on the nature and severity of the disease to be prevented or treated, the individual condition of the patient or animal, the route of administration, and the dosage form. Generally speaking, the appropriate daily dosage range of the compounds of this invention is 0.001-5 mg/kg body weight. The above dosage can be administered as a single dosage unit or divided into several dosage units, depending on the clinical experience of the physician and the dosing regimen, including the use of other therapeutic means.

The compounds or compositions of this invention can be taken alone or in combination with other therapeutic drugs or symptomatic medications. When the compounds of this invention have a synergistic effect with other therapeutic drugs, their dosage should be adjusted according to the actual situation.

The fourth aspect of the technical solution of this invention is to provide the use of the methyl ophiopogonone A class derivatives and their pharmaceutically acceptable salts as described in the first aspect, as well as the pharmaceutical compositions as described in the third aspect, in the preparation of drugs for the prevention and/or treatment of cardiovascular-related diseases. The cardiovascular-related diseases referred to herein are selected from one or more of the following: myocardial ischemia, myocardial injury, myocardial hypertrophy, coronary heart disease, hypertension, heart failure, cardiomyopathy, arrhythmia, myocardial infarction, and angina pectoris.

### Beneficial Technical Effects

The methyl ophiopogonone A class derivatives of this invention have demonstrated significant cardioprotective activity against myocardial cell injury in various models, including isoproterenol-induced myocardial cell injury, hypoxia-reoxygenation-induced myocardial cell injury, and angiotensin II-induced myocardial cell injury. These derivatives significantly enhance myocardial cell survival, showing good efficacy at a concentration as low as 0.1 μM. In the angiotensin II-induced myocardial cell hypertrophy model, they also exhibit significant inhibitory activity against myocardial cell hypertrophy, with good efficacy observed at 0.1 μM. Myocardial injury and hypertrophy are critical pathological processes associated with the development of cardiovascular diseases, including hypertension-induced heart failure. Multiple experimental results indicate that the compounds of this invention possess robust cardioprotective and anti-hypertrophy activities, which in most cases, outperform methyl ophiopogonone A and diltiazem under the experimental conditions.

In in vivo models, the methyl ophiopogonone A class derivatives I and II have demonstrated the following significant activities: In the classic isoproterenol-induced ischemic heart disease mouse model, they exhibit clear therapeutic effects on myocardial ischemia and hypertrophy. They reduce the levels of heart failure/myocardial hypertrophy biomarkers NT-proBNP and ANP, lower serum myocardial enzyme CK-MB levels, alleviate calcium overload in myocardial tissue, and significantly improve myocardial tissue pathological damage. In the angiotensin II-induced hypertensive heart disease mouse model, they show clear antihypertensive and cardioprotective effects. They significantly reduce the progressive increase in systolic blood pressure induced by angiotensin II, lower the levels of heart failure/myocardial hypertrophy biomarker BNP, reduce serum myocardial injury biomarkers troponin cTn-T and myocardial enzyme CK-MB levels, decrease the production and release of inflammatory factor TNF-α, mitigate the overactivation effects of the renin-angiotensin-aldosterone system (RAAS) and sympathetic nervous system, improve myocardial hypertrophy, enhance left ventricular systolic and diastolic functions, and increase left ventricular compliance. In in vivo animal models, the methyl ophiopogonone A class derivatives of this invention show superior activity compared to methyl ophiopogonone A, diltiazem, and spironolactone, with activity comparable to that of telmisartan, but without the adverse effect of hypotension caused by telmisartan.

Considering the in vitro and in vivo experimental results, the methyl ophiopogonone A class derivatives of this invention hold potential value for the development of anti-cardiovascular disease drugs.

### BRIEF DESCRIPTION OF THE DRAWINGS

FIG. 1. Illustration of the improvement of myocardial cell hypertrophy damage caused by Angiotensin II (AngII) by methyl ophiopogonone A class derivatives I and II.

FIG. 2. Illustration of the improvement of pathological changes in myocardial ischemia/myocardial hypertrophy in mouse heart tissue induced by isoproterenol (ISO) by methyl ophiopogonone A class derivatives I and II (H. E. 200×).

FIG. 3. Illustration of the effects of methyl ophiopogonone A class derivatives I and II on M-mode echocardiography in mice with hypertensive heart disease induced by Angiotensin II (AngII).

### SPECIFIC IMPLEMENTATION METHODS

This invention provides three types of methyl ophiopogonone A class derivatives for the treatment of cardiovascular diseases, as well as their preparation and application. The following examples are provided to further illustrate the invention. These examples are for explanatory and illustrative purposes only and do not in any way limit the scope of the invention.

The structure of the compounds was determined by nuclear magnetic resonance hydrogen spectrum (1H NMR) and/or mass spectrometry (MS). NMR chemical shifts (8) are given in units of 10\hat{ }-6 (ppm). NMR measurements were performed using a Bruker AV400 NMR spectrometer. MS measurements were conducted using a Thermo Scientific (ESI) mass spectrometer.

Column chromatography generally uses silica gel with a mesh size of 200-300 as the carrier.

The known starting materials of this invention can be used or synthesized according to known methods in the field, or can be purchased from companies such as Coupling Technology, Bellingham&Stanley, and Anaiji Chemistry.

Hydrogen atmosphere refers to the reaction flask being connected to a hydrogen gas balloon with a volume of about 1 L.

In the following pharmacological research examples, experimental methods not specifically mentioned are carried out according to routine methods and conditions, or selected according to the product instructions.

In the following examples, some substances or abbreviations correspond to the following full names:
Control: Blank control
Model: Model control
ISO: Isoproterenol
H/R: Hypoxia-reoxygenation
Ang II: Angiotensin I
DMSO: Dimethyl sulfoxide
NaH: Sodium hydride
Na₂SO₄: Sodium sulfate
NaCl: Sodium chloride
SFC: Supercritical fluid chromatography
DEA: Diethylamine

### Example 1 Preparation of Methyl Ophiopogonone A Class Derivative I

### 1. Preparation of Compound (2) (1-(2-hydroxy-4,6-bis(methoxymethoxy)phenyl)ethan-1-one)

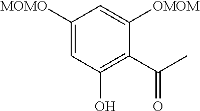

A mixture of 2, 4, 6-trihydroxyacetophenone (10.0 g, 59.5 mmol) and N, N-diisopropylethylamine (23.1 g, 178.5 mmol) was dissolved in dichloromethane (200 mL). Bromomethoxymethyl ether (10.7 mL, 130.9 mmol) was added dropwise under an ice bath over 2 hours. The reaction progress was monitored by UPLC-MS. After complete consumption of the raw material, the reaction was quenched with methanol. The mixture was concentrated under reduced pressure, and the residue was purified by silica gel column chromatography using a petroleum ether/ethyl acetate eluent (10:1). The title compound was obtained with a yield of 52% (8.0 g).

¹H NMR (400 MHZ, CDCl₃) δ 13.72 (s, 1H), 6.26 (dd, J=9.3, 2.3 Hz, 2H), 5.26 (s, 2H), 5.17 (s, 2H), 3.52 (s, 3H), 3.47 (s, 3H), 2.66 (s, 3H).

### 2. Preparation of compound (3) (3-(benzo[d][1,3]dioxol-5-yl)-1-(2-hydroxy-4, 6-bis methoxymethoxy)phenyl) prop-2-en-1-one)

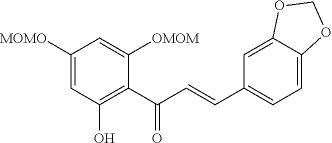

A solution of the compound of Formula (2) (8.0 g, 31.2 mmol) in N,N-dimethylformamide was cooled in an ice-bath. Sodium hydride (NaH, 1.1 g, 46.8 mmol) was added slowly, followed piperonal (4.7 g, 31.2 mmol). The reaction mixture was stirred under the ice bath for 30 minutes. The reaction progress was monitored by UPLC-MS. After complete consumption of the raw material, water was added to the reaction mixture and extracted with ethyl acetate. The organic phase was washed with water and saturated NaCl solution, and then concentrated under reduced pressure. The residue was purified by silica gel column chromatography using a petroleum ether/ethyl acetate eluent (5:1). The title compound was obtained with a yield of 74% (9.0 g).

¹H NMR (400 MHZ, CDCl₃) δ 13.89 (s, 1H), 7.82-7.69 (m, 2H), 7.12-7.07 (m, 2H), 6.84 (d, J=7.9 Hz, 1H), 6.28 (dd, J=28.0, 2.3 Hz, 2H), 6.03 (s, 2H), 5.29 (s, 2H), 5.19 (s, 2H), 3.54 (s, 3H), 3.49 (s, 3H).

### 3. Preparation of compound (4) (3-(benzo[d][1,3]dioxol-5-yl)-1-(2-hydroxy-4, 6-bis(methoxymethoxy)phenyl) propan-1-one)

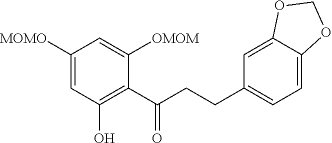

A solution of the compound of Formula (3) (9 g, 23.2 mmol) in tetrahydrofuran (150 mL) was treated with palladium on carbon (Pd/C, 0.9 g). The mixture was stirred under a hydrogen atmosphere at room temperature for 16 hours. The reaction progress was monitored by UPLC-MS. After complete consumption of the raw material, the solid was filtered out and the filtrate was concentrated under reduced pressure to obtain the title compound with a yield of 79% (7.2 g).

¹H NMR (400 MHZ, CDCl₃) δ 13.68 (s, 1H), 7.26 (s, 1H), 6.77-6.65 (m, 3H), 6.27 (dd, J=11.6, 2.3 Hz, 2H), 5.93 (s, 2H), 5.24 (s, 2H), 5.17 (s, 2H), 3.48 (d, J=3.1 Hz, 7H), 3.35-3.29 (m, 2H), 2.94 (t, J=7.6 Hz, 2H).

### 4. Preparation of compound (1) (3-(benzo[d][1,3]dioxol-5-yl)-1-(2-hydroxy-4, 6-bis(methoxymethoxy)phenyl) propan-1-one)

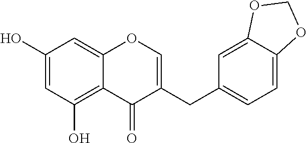 [SMILES: O=c1c(Cc2ccc3c(c2)OCO3)coc2cc(O)cc(O)c12]

A solution of the compound of Formula (4) (300 mg, 0.76 mmol) was dissolved in 5 ml of ethyl acetate. Under a nitrogen atmosphere, N, N-dimethylformamide (224 mg, 3.06 mmol) and phosphorus oxychloride (470 mg, 3.06 mmol) were slowly added dropwise to the solution. The reaction mixture was stirred at room temperature for 5 hours. The reaction progress was monitored by UPLC-MS. After the complete consumption of raw material, the reaction was quenched with water and extracted with ethyl acetate (3×25 mL). The combined organic phase were washed with water and saturated NaCl solution, then dried over anhydrous Na2SO4. Following filtration, and the filtrate was concentrated under reduced pressure. The residue was purified by silica gel column chromatography using a hexane/ethyl acetate eluent (1:1) to obtain the title compound with a yield of 24.5% (34 mg).

¹H NMR (400 MHz, DMSO-d₆) δ 12.70 (s, 1H), 11.36-11.08 (m, 1H), 8.19 (s, 1H), 6.90-6.73 (m, 3H), 6.31-6.30 (m, 1H), 6.16-6.15 (m, 1H), 5.95 (s, 2H), 3.57 (s, 2H).

MS (ESI−): [(M−H)⁻]311.0.

HPLC Purity: 99.34% (254 nm), 99.13% (214 nm).

### Example 2: Preparation of Methyl Ophiopogonone A Class Derivatives II and III

### 1. Preparation of compound (5) (1-(2-hydroxy-4,6-bis(methoxymethoxy)phenyl) ethan-1-one)

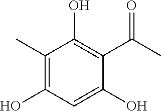

2, 4, 6-trihydroxytoluene (5 g, 35.70 mmol) was dissolved in 100 ml glacial acetic acid. Boron trifluoride diethyl etherate (6.08 g, 42.80 mmol) and acetic anhydride (4.37 g, 42.80 mmol) were added at room temperature. The reaction was carried out at 100° C. under N₂ for 5 hours. After cooling, the acetic acid was removed under reduced pressure, and ethyl acetate was added. The organic phase was washed with water and saturated NaCl solution, dried over anhydrous Na₂SO₄, filtered, and the filtrate was concentrated under reduced pressure. The residue was purified by silica gel column chromatography using a petroleum ether/ethyl acetate eluent (5:1) to obtain the title compound with a yield of 62% (4.0 g). MS (ESI+): [(M+H)⁺] 183.1.

### 2. Preparation of Compound (6) (1-(2,4,6-tris(methoxymethoxy)-3-methylphenyl) ethan-1-one)

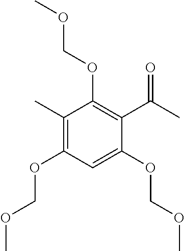 [SMILES: COCOc1cc(OCOC)c(C(C)=O)c(OCOC)c1C]

A solution of the compound of Formula (5) (4.0 g, 22.0 mmol) in THF (60 mL) was cooled in an ice bath. Sodium hydride (NaH, 3.70 g, 154.0 mmol) was added slowly, followed by the dropwise addition of bromomethoxymethyl ether (16.50 g, 132.0 mmol). The reaction mixture was stirred under the ice bath for 3 hours. Water and ethyl acetate were slowly added to quench the reaction, then extracted with ethyl acetate (3×20 mL). The combined organic phase was washed with water and saturated NaCl solution, and then concentrated under reduced pressure. The residue was purified by silica gel column chromatography using a petroleum ether/ethyl acetate eluent (5:1) to obtain the title compound with a yield of 16% (1.13 g).

¹H NMR (400 MHz, DMSO-d₆) δ 6.72 (s, 1H), 5.23 (s, 2H), 5.18 (s, 2H), 4.86 (s, 2H), 3.40 (s, 3H), 3.39 (s, 3H), 3.36 (s, 3H), 2.42 (s, 3H), 2.04 (s, 3H).

MS (ESI+): [(M+H)+] 315.1.

### 3. Preparation of Compound (7) (E)-4-(benzo[d][1,3]dioxol-5-yl)-1-(2, 4, 6-tris(methoxymethoxy)-3-methylphenyl) but-2-en-1-one)

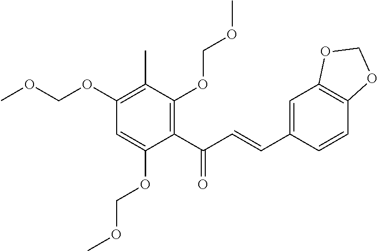 [SMILES: COCOc1cc(OCOC)c(C(=O)/C=C/c2ccc3c(c2)OCO3)c(OCOC)c1C]

A solution of the compound of Formula (6) (1.13 g, 3.6 mmol) in of tetrahydrofuran (20 mL) was cooled in an ice bath. Sodium hydride (NaH, 130 mg, 5.4 mmol) and piperonal (0.54 g, 3.6 mmol) were slowly added. The reaction mixture was stirred under the ice bath for 1 hour. Water and ethyl acetate were slowly added to quench the reaction, followed by extraction with ethyl acetate (3×20 mL). The combined organic phase were washed with water and saturated NaCl solution, and then concentrated under reduced pressure. The residue was purified by silica gel column chromatography using a petroleum ether/ethyl acetate eluent (5:1) to obtain the title compound with a yield of 58% (1.1 g).

¹H NMR (400 MHz, DMSO-d₆) δ 7.41 (s, 1H), 7.22 (d, J=16.0 Hz, 1H), 7.16 (d, J=8.1 Hz, 1H), 6.94 (dd, J=11.9, 9.6 Hz, 2H), 6.76 (s, 1H), 6.08 (s, 2H), 5.26 (s, 2H), 5.13 (s, 2H), 4.85 (s, 2H), 3.48 (s, 1H), 3.43 (s, 3H), 3.39 (d, J=5.3 Hz, 1H), 3.33 (s, 3H), 3.27 (s, 3H), 2.08 (s, 3H).

MS (ESI+): [(M-13)+] 447.3.

### 4. Preparation of Compound (8) (4-(benzo[d][1,3]dioxol-5-yl)-1-(2, 4, 6-tris(methoxymethoxy)-3-methylphenyl) butan-1-one)

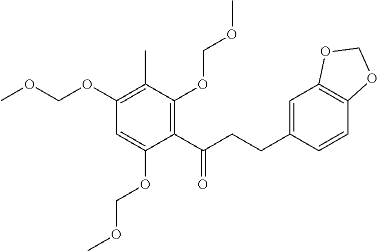 [SMILES: COCOc1cc(OCOC)c(C(=O)CCc2ccc3c(c2)OCO3)c(OCOC)c1C]

A solution of the compound of Formula (7) (400 mg, 0.87 mmol) in tetrahydrofuran (10 ml) was treated with Pd/C (462 mg, 4.34 mmol) under a hydrogen atmosphere at room temperature for 8 hours. The reaction progress was monitored by UPLC-MS. After complete consumption of the raw material, the solid was filtered out and the filtrate was concentrated under reduced pressure. The residue was purified by silica gel column chromatography using a petroleum ether/ethyl acetate eluent (10:1) to obtain the title compound with a yield of 50% (200 mg).

¹H NMR (400 MHz, DMSO-d₆) δ 6.83-6.79 (m, 2H), 6.71-6.68 (m, 2H), 5.95 (s, 2H), 5.23 (s, 2H), 5.13 (s, 2H), 4.80 (s, 2H), 3.40-3.39 (m, 5H), 3.36 (s, 3H), 3.32 (s, 3H), 3.02 (t, J=7.4 Hz, 2H), 2.82 (t, J=7.4 Hz, 2H), 2.03 (s, 3H). MS (ESI+): [(M-13)⁺] 449.2.

### 5. Preparation of Compounds (II) and (III) (3-(benzo[d][1,3]dioxol-5-ylmethyl)-5,7-dihydroxy-8-methyl-4H-chromen-4-one (II) and (benzo[d][1,3]dioxol-5-ylmethyl)-5,7-dihydroxy-6-methyl-4H-chromen-4-one (III))

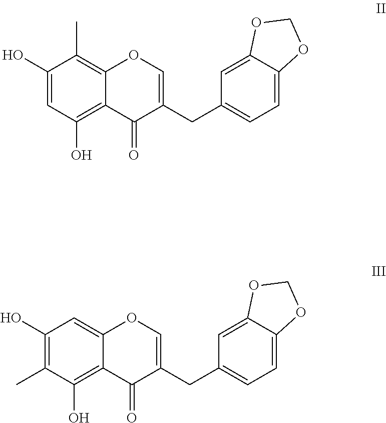 [SMILES: Cc1c(O)cc(O)c2c(=O)c(Cc3ccc4c(c3)OCO4)coc12 ; Cc1c(O)cc2occ(Cc3ccc4c(c3)OCO4)c(=O)c2c1O]

A solution of the compound of Formula (8) (200 mg, 0.43 mmol) in ethyl acetate (10 mL) was treated with. N, N-dimethylformamide (70 mg, 0.95 mmol) and phosphorus oxychloride (663 mg, 4.32 mmol) under nitrogen atmosphere at 0° C. The reaction mixture was warmed to room temperature and stirred for 5 hours. The reaction progress was monitored by UPLC-MS. After complete consumption of the raw material, the reaction was quenched with water, extracted with ethyl acetate (3×10 mL). The combined organic phase were concentrated under reduced pressure. The residue was purified by preparative HPLC to obtain a mixture of compounds with structures (II) and (III). Further purification by SFC (mobile phase: CO₂/MeOH (0.1% DEA)=65/35) yielded the title compound (II) (t_{R}=1.073 min, 20 mg, yield 14%) and the title compound (III) (t_{R}=0.903 min, 9 mg, yield 6%).

II: ¹H NMR (400 MHz, DMSO-d₆) δ 12.62 (s, 1H), 10.79 (s, 1H), 8.27 (s, 1H), 6.87 (d, J=1.2 Hz, 1H), 6.82-6.80 (m, 1H), 6.77-6.73 (m, 1H), 6.29 (s, 1H), 5.95 (s, 2H), 3.58 (s, 2H), 2.06 (s, 3H).

MS (ESI+): [(M+H)⁺] 327.0.

HPLC Purity: 99.71% (254 nm), 99.33% (214 nm)

III: ¹H NMR (400 MHz, DMSO-d₆) δ 12.96 (s, 1H), 10.86 (s, 1H), 8.19 (s, 1H), 6.87 (d, J=1.4 Hz, 1H), 6.82-6.80 (m, 1H), 6.76-6.74 (m, 1H), 6.42 (s, 1H), 5.95 (s, 2H), 3.59 (s, 2H), 1.96 (s, 3H).

MS (ESI+): [(M+H)⁺] 327.0.

HPLC Purity: 99.48% (254 nm), 98.77% (214 nm)

### PHARMACOLOGICAL EXPERIMENTS

### Experimental Example 1: Evaluation of the Example Compounds Using an Isoproterenol (ISO)-Induced Myocardial Cell Injury Model

### Experimental Methods:

Rat H9C2 cardiomyocytes in the logarithmic growth phase were seeded into 96-well plates at a density of 7×10⁴ cells/mL, with 100 μL per well. The cells were divided into the following groups: blank control group, ISO model group, ISO+diltiazem 10 μM group, ISO+Formula I 0.1 μM group, ISO+Formula I 0.3 μM group, ISO+Formula I 1 μM group, ISO+Formula I 3 μM group, ISO+Formula I 5 μM group, ISO+Formula I 10 μM group, ISO+Formula II 0.1 μM group, ISO+Formula II 0.3 μM group, ISO+Formula II 1 μM group, ISO+Formula II 3 μM group, ISO+Formula II 5 μM group, ISO+Formula II 10 μM group, ISO+Formula III 1 μM group, ISO+Formula III 5 μM group, ISO+Formula III 10 μM group, ISO+methyl ophiopogonone A 1 μM group, ISO+methyl ophiopogonone A 5 μM group, and ISO+methyl ophiopogonone A 10 μM group. After 24 hours of culture, the drug groups were treated with 800 μM ISO and the corresponding concentrations of drugs. The ISO model group received 800 μM ISO and an equal volume of DMSO, and the blank control group received an equal volume of DMSO. The cells were then cultured for another 24 hours. After removing the supernatant, 100 μL MTT solution (0.5 mg/mL) was added to each well and incubated in the cell culture incubator for 4 hours. After removing the supernatant, 150 μL DMSO was added to each well, mixed by shaking for 10 minutes, and the absorbance was measured at 570 nm using a microplate reader to calculate cell viability. Cell viability (%)=[(As−Ab)/(Ac−Ab)]×100%. As: Experimental well (medium with cells, MTT, ISO); Ac: Control well (medium with cells, MTT, without ISO); Ab: Blank well (medium without cells, MTT, without ISO).

### Experimental Results:

The results are shown in Table 1. Compared with the blank control group, the survival rate of H9C2 cardiomyocytes treated with ISO for 24 h was significantly decreased (54.71%), and the OD value was statistically different from that of the blank control group. Compared with the ISO model group, compounds of formula I (0.1-10 μM), formula II (0.1-10 μM) and formula III (1-10 μM) showed significant protective activity against ISO-induced myocardial cell injury, and could significantly improve the cell survival rate, and the OD value was statistically different from that of the model group. methyl ophiopogonone A at 1 μM and 5 μM also showed a significant protective effect on ISO-injured cardiomyocytes. Compared with methyl ophiopogonone A at the same dose, compounds of Formula I and Formula II showed better activity, while compounds of Formula III showed comparable activity to methyl ophiopogonone A. Diltiazem 10 μM also had protective activity against ISO-induced myocardial cell injury. Compounds of Formula I and II had better protective activity against ISO-induced myocardial cell injury than diltiazem. Compounds of Formula III 1 μM had better protective activity than diltiazem 10 μM.

TABLE 1
Protective effects of example compounds against isoproterenol
(ISO)-induced myocardial cell injury

| | dose | | Cell survival rate |
|---|---|---|---|
| Group | (μM) | OD | (%) |
| Control | — | 1.209 ± 0.033 | 100.00 |
| ISO model | — | 0.661 ± 0.055*** | 54.71 |
| Diltiazem | 10 | 0.773 ± 0.029^{#} | 63.94 |
| Formula I | 0.1 | 0.983 ± 0.144^{###} | 81.29 |
| | 0.3 | 0.929 ± 0.105^{##} | 76.84 |
| | 1 | 0.969 ± 0.065^{###&&} | 73.98 |
| | 3 | 0.926 ± 0.045^{###} | 76.59 |
| | 5 | 0.807 ± 0.088^{##} | 66.71 |
| | 10 | 0.839 ± 0.091^{###&} | 69.43 |
| Formula II | 0.1 | 0.964 ± 0.132^{##} | 79.76 |
| | 0.3 | 0.964 ± 0.031^{###} | 79.77 |
| | 1 | 0.904 ± 0.072^{###&&} | 74.78 |
| | 3 | 0.963 ± 0.064^{###} | 79.64 |
| | 5 | 0.784 ± 0.067^{##} | 64.88 |
| | 10 | 0.806 ± 0.067^{###} | 66.65 |
| Formula III | 1 | 0.815 ± 0.041^{###} | 67.39 |
| | 5 | 0.792 ± 0.061^{##} | 65.51 |
| | 10 | 0.750 ± 0.065^{#} | 62.06 |
| methyl | 1 | 0.781 ± 0.029^{##} | 64.63 |
| ophiopogonone A | | | |
| | 5 | 0.801 ± 0.045^{##} | 66.28 |
| | 10 | 0.724 ± 0.024^{(p = 0.09)} | 59.89 |

***P < 0.001 compared with the control group;
^{#}P < 0.05, ^{##}P < 0.01, ^{###}P < 0.001 compared with the model group;
^{&}P < 0.05, ^{&&}P < 0.01 compared with the methyl ophiopogonone A group at the same dose.
Note:
The doses of the drugs used had no toxicity to H9C2 cardiomyocytes. The data are not shown, the same below.

### Experimental Example 2: Evaluation of Compounds Using an Oxygen Deprivation and Reoxygenation (H/R)-Induced Cardiomyocyte Injury Model Experimental Methods

H9C2 cardiomyocytes in the logarithmic growth phase were seeded into 96-well plates at a density of 7×10⁴ cells/mL, with 100 μL per well. The groups were divided as follows: blank control group, H/R model group, H/R+Diltiazem 10 μM group, H/R+Formula I 0.1 μM group, H/R+Formula I 0.3 μM group, H/R+Formula I 1 μM group, H/R+Formula I 3 μM group, H/R+Formula I 5 μM group, H/R+Formula I 10 μM group, H/R+Formula II 0.1 μM group, H/R+Formula II 0.3 μM group, H/R+Formula II 1 μM group, H/R+Formula II 3 μM group, H/R+Formula II 5 μM group, H/R+Formula II 10 μM group, H/R+Formula III 0.1 μM group, H/R+ Formula III 0.3 μM group, H/R+Formula III 1 μM group, H/R+Formula III 3 μM group, H/R+Formula III 5 μM group, H/R+Formula III 10 μM group, H/R+Methyl Ophiopogonone A 1 μM group, H/R+Methyl Ophiopogonone A 5 μM group, and H/R+Methyl Ophiopogonone A 10 μM group.

After 24 hours of culture, the culture medium of the H/R and drug-treated groups was replaced with serum-free glucose-free DMEM medium (100 μL per well). The cells were then placed in an anaerobic incubator and cultured under hypoxic conditions in a 37° C. cell culture incubator. After 6 hours, the supernatant of the H/R and drug-treated groups was removed and replaced with high-glucose medium containing 10% fetal bovine serum (100 μL per well). Each drug-treated group received the corresponding concentration of the drug, while the H/R model group and the blank control group received an equal volume of DMSO. The cells were then reoxygenated in a cell culture incubator at 37° C., 95% 02, and 5% CO₂ for 12 hours.

After removing the supernatant, 100 μL of CCK solution (the volume of CCK accounted for 10% of the DMEM culture medium volume) was added to each well. The cells were cultured in the cell culture incubator for 1 hour, and the absorbance was measured at 450 nm using a microplate reader to calculate the cell viability. Cell viability was calculated as follows: Cell viability (%)=(Experimental well-Blank well)/(Control well-Blank well)×100%.

### Experimental Results:

The results are shown in Table 2. Compared with the blank control group, reoxygenation after oxygen-glucose deprivation led to significant damage to cardiomyocytes, and the cell survival rate was only 50.80%. Compounds of Formula I (0.1, 0.3, 1, 3, 10 μM), compounds of Formula II (0.1, 0.3, 3 μM) and compounds of Formula III (3 μM) showed significant protective activities against hypoxia-reoxygenation-induced cardiomyocyte damage, significantly increasing the survival rate of hypoxia-reoxygenation-damaged cardiomyocytes. There were statistically significant differences in the OD values compared with those of the model group. methyl ophiopogonone A at a concentration of 10 μM also showed significant protective activity against hypoxia-reoxygenation-induced myocardial damage, while methyl ophiopogonone A at 1 μM and 5 μM did not show significant protective activity against hypoxia-reoxygenation-induced myocardial damage. Diltiazem at 10 μM did not show significant protective effect against hypoxia-reoxygenation-induced myocardial damage. In this model, the activities of compounds of Formula I and Formula II were superior to that of methyl ophiopogonone A, and they showed effects at low doses. The activities of compounds of Formula I, Formula II and Formula III were superior to that of Diltiazem. Compounds of Formula I, Formula II and Formula III showed activities against ischemia-reperfusion-induced cardiomyocyte damage, and the activities of compounds of Formula I and Formula II were superior to that of Formula III compounds.

TABLE 2
Protective effects of the compounds of the examples against
hypoxia-reoxygenation (H/R)-induced cardiomyocyte damage.

| | Dosage | | Cell viability |
|---|---|---|---|
| Group | (μM) | OD | (%) |
| Control | — | 1.040 ± 0.044 | 100.00 |
| H/R model | — | 0.528 ± 0.032*** | 50.80 |
| diltiazem | 10 | 0.522 ± 0.061 | 50.17 |
| Formula I | 0.1 | 0.630 ± 0082^{###} | 60.56 |
| | 0.3 | 0.628 ± 0.061^{###} | 60.34 |
| | 1 | 0.576 ± 0.063^{##} | 55.39 |
| | 3 | 0.620 ± 0.069^{###} | 59.62 |
| | 5 | 0.566 ± 0.074^{(p = 0.07)} | 54.42 |
| | 10 | 0.575 ± 0.091^{#} | 55.26 |
| Formula II | 0.1 | 0.630 ± 0.070^{###} | 60.54 |
| | 0.3 | 0.612 ± 0.059^{###} | 58.84 |
| | 1 | 0.542 ± 0.075 | 52.14 |
| | 3 | 0.640 ± 0.070^{###} | 61.54 |
| | 5 | 0.466 ± 0.023 | 44.78 |
| | 10 | 0.498 ± 0.071 | 47.86 |
| Formula III | 0.1 | 0.552 ± 0.067 | 53.11 |
| | 0.3 | 0.500 ± 0.062 | 48.09 |
| | 1 | 0.555 ± 0.053 | 53.35 |
| | 3 | 0.580 ± 0.069^{#} | 55.80 |
| | 5 | 0.543 ± 0.037 | 52.20 |
| | 10 | 0.527 ± 0.047 | 50.65 |
| methyl | 1 | 0.540 ± 0.034 | 51.93 |
| ophiopogonone A | | | |
| | 5 | 0.501 ± 0.042 | 48.17 |
| | 10 | 0.581 ± 0.054^{#} | 55.82 |

***P < 0.001 compared with the control group;
^{#}P < 0.05, ^{##}P < 0.01, ^{###}P < 0.001 compared with the model group.

### Experimental Example 3: Detection Experiment of the Compounds in the Examples Using a Cardiomyocyte Injury Model Induced by Angiotensin II (Ang II)

### Experimental Method:

Rat H9C2 cardiomyocytes in the logarithmic growth phase were seeded at a density of 7×10⁴ cells/mL into 96-well plates, with 100 μL per well. The cells were divided into a blank control group, an Ang II model group, an Ang II+diltiazem 10 μM group, an Ang II+Formula I 0.1 μM group, an Ang II+Formula I 0.3 μM group, an Ang II+Formula I 1 μM group, an Ang II+Formula I 3 μM group, an Ang II+Formula I 10 μM group, an Ang II+Formula II 0.1 μM group, an Ang II+Formula II 0.3 μM group, an Ang II+Formula II 1 μM group, an Ang II+Formula II 3 μM group, an Ang II+Formula II 10 μM group, and an Ang II+methyl ophiopogonone A 10 μM group. After 24 hours of culture, the drug treatment groups were respectively added with 50 μM Ang II and the corresponding concentration of the drug. The Ang II model group was added with 50 μM Ang II and an equal volume of DMSO, and the blank control group was added with an equal volume of DMSO. Then, the cells were cultured for another 24 hours. After removing the supernatant, 100 μL of MTT solution (0.5 mg/mL) was added to each well, and the plates were placed in an incubator for 4 hours. After removing the supernatant again, 150 μL of DMSO was added to each well, and the mixture was shaken and mixed evenly for 10 minutes. The absorbance was measured at 570 nm using a microplate reader to calculate the cell viability.

Cell viability (%)=[(As−Ab)/(Ac−Ab)]×100%
As: Experimental well (culture medium containing cells, MTT, Ang II);
Ac: Control well (culture medium containing cells, MTT, without Ang II);
Ab: Blank well (culture medium without cells, MTT, without Ang II).

### Experimental Results:

The results are shown in Table 3. Compared with the blank control group, 50 μM AngII caused significant damage to cardiomyocytes. The cell survival rate was only 70.81%, and there was a significant difference in the OD value compared with the blank control group. Compared with the model group, compound I (3, 10 μM) and compound II (0.1, 1, 10 μM) showed significant protective activities against cardiomyocyte damage induced by AngII. They significantly increased the cell survival rate, and there were statistical differences in the OD value compared with the model group. Other dosage groups of compound I and compound II also showed a trend of improving cardiomyocyte damage induced by AngII. 10 μM methyl ophiopogonone A and 10 μM diltiazem also showed significant protective activities against cardiomyocyte damage induced by AngII. In the cardiomyocyte damage model induced by AngII, the activities of compound I and compound II were comparable to those of methyl ophiopogonone A and diltiazem. The activity of compound II at a dosage of 0.1 μM was slightly better than that of methyl ophiopogonone A at 10 μM.

TABLE 3
Protective effects of the compounds in the examples against
cardiomyocyte damage induced by angiotensin II (AngII)

| | | | Cell viability |
|---|---|---|---|
| Group | Dosage (μM) | OD | (%) |
| Control | — | 1.076 ± 0.063 | 100.00 |
| AngII model | — | 0.762 ± 0.022*** | 70.81 |
| diltiazem | 10 | 0.857 ± 0.042^{###} | 79.67 |
| Formula I | 0.1 | 0.809 ± 0.060 | 75.14 |
| | 0.3 | 0.780 ± 0.046 | 72.48 |
| | 1 | 0.791 ± 0.043 | 73.51 |
| | 3 | 0.816 ± 0.035^{##} | 75.86 |
| | 10 | 0.823 ± 0.048^{##} | 76.43 |
| Formula II | 0.1 | 0.876 ± 0.044^{###} | 81.36 |
| | 0.3 | 0.791 ± 0.062 | 73.49 |
| | 1 | 0.835 ± 0.052^{##} | 77.58 |
| | 3 | 0.785 ± 0.048 | 72.98 |
| | 10 | 0.814 ± 0.053^{#} | 75.59 |
| methyl | 10 | 0.833 ± 0.041^{##} | 77.40 |
| ophiopogonone A | | | |

*** P < 0.001 compared with the control group;
^{#}P < 0.05, ^{##}P < 0.01, ^{###}P < 0.001 compared with the model group.

### Experimental Example 4: Detection Experiment of the Compounds in the Examples Using a Cardiomyocyte Hypertrophy Model Induced by Angiotensin II (Ang II)

### Experimental Method:

Adjust the H9C2 cardiomyocytes in the logarithmic growth phase to a density of 1.2×10⁵ cells/well and inoculate them into six-well plates. The cells are divided into a blank control group, an Ang II model group, an Ang II+diltiazem 10 μM group, an Ang II+Formula I 0.1 μM group, an Ang II+Formula I 0.5 μM group, an Ang II+Formula I 1 μM group, an Ang II+Formula II 0.1 μM group, an Ang II+Formula II 0.5 μM group, and an Ang II+Formula II 1 μM group. After culturing for 24 hours, the drug administration groups are added with 40 μM Ang II and the corresponding concentrations of drugs. The Ang II+diltiazem 10 μM group is added with 40 μM Ang II and 10 μM diltiazem. The Ang II model group is added with 40 μM Ang II and an equal volume of DMSO. The blank control group is added with an equal volume of DMSO. Then, continue culturing for 24 hours. Discard the culture medium, wash the cells 3 times with PBS. Add 1 mL of methanol to each well and fix the cells at −20° C. for 20 minutes, and then let them return to room temperature. After discarding the methanol, add 1 mL of 0.5% crystal violet staining solution to each well and stain for 10 minutes. Wash the cells with clean water and take pictures under an inverted microscope. Randomly select fields of view for photographing. Use Image J software to measure the sum of the surface areas of all cardiomyocytes in the field of view and count the number of cells in the field of view at the same time to obtain the surface area of each cardiomyocyte. The surface area of a single cardiomyocyte=the sum of the surface areas of all cardiomyocytes in the field of view/the number of cardiomyocytes in the field of view.

### Experimental Results:

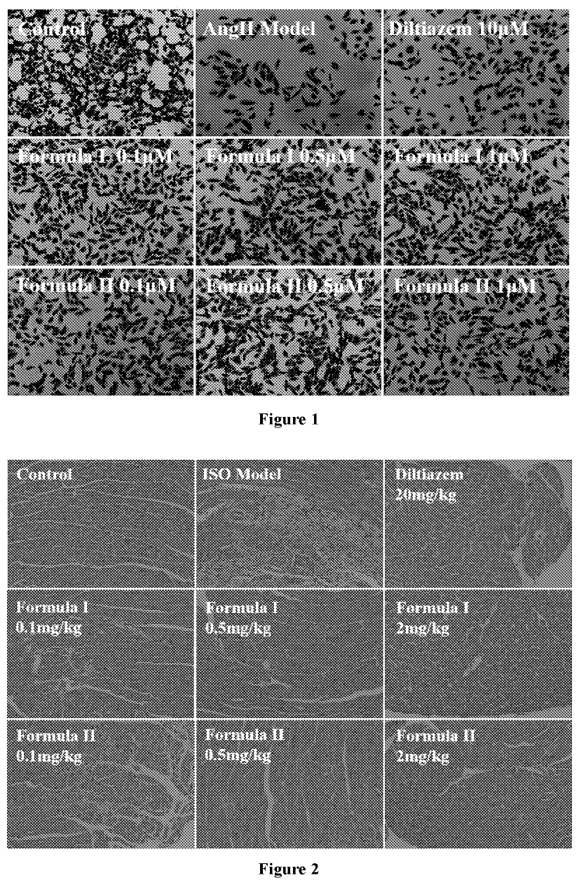

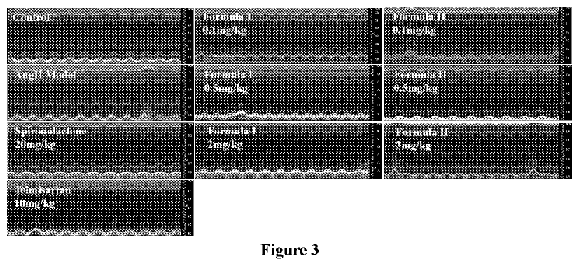

The results are shown in FIG. 1 and Table 4. After treating H9C2 cells with 40 μM of AngII for 24 hours, compared with the blank control group, the number of cardiomyocytes in the model group decreased significantly, and the volume of cardiomyocytes increased significantly. Compounds of Formula I at 0.1 μM, 0.5 μM, and 1 μM, and compounds of Formula II at 0.1 μM, 0.5 μM, and 1 μM could all significantly improve the decrease in cell number caused by AngII, and the number of cells increased significantly. The results of the surface area of a single cardiomyocyte are shown in Table 4. The surface area of a single cardiomyocyte in the model group increased significantly compared with that in the blank control group, showing a statistically significant difference. Compounds of Formula I at 0.1 μM, 0.5 μM, and 1 μM and compounds of Formula II at 0.1 μM, 0.5 μM, and 1 μM could all significantly inhibit the cardiomyocyte hypertrophy caused by AngII. The surface area of a single cardiomyocyte was significantly lower than that in the model group. Diltiazem at 10 μM also significantly reduced the cardiomyocyte hypertrophy caused by AngII. Compounds of Formula I and Formula II at doses of 0.1 μM, 0.5 μM, and 1 μM showed better inhibitory activity against cardiomyocyte hypertrophy than the positive drug diltiazem at 10 μM.

TABLE 4
Effects of the compounds in the examples of implementation on
cardiomyocyte hypertrophy-surface area of a single cardiomyocyte (μM²)

| | | | | | | | Myocardial |
|---|---|---|---|---|---|---|---|
| | Con. | View | View | View | View | View | surface |
| Group | (μM) | 1 | 2 | 3 | 4 | 5 | area(μm²) |
| Control | — | 157.86 | 220.78 | 216.49 | 178.22 | 161.18 | 186.91 ± 26.85 |
| Model | — | 623.16 | 654.51 | 644.21 | 646.13 | 774.26 | 668.45 ± 53.90*** |
| diltiazem | 10 | 406.26 | 402.67 | 411.02 | 359.20 | 380.97 | 392.02 ± 19.37^{###} |
| Formula | 0.1 | | | 344.78 | 338.55 | | |
| I | | 362.68 | 351.69 | | | 410.64 | 361.67 ± 25.76^{###} |
| | 0.5 | 404.40 | 360.65 | 322.34 | 283.2 | 334.96 | 341.11 ± 40.33^{###} |
| | 1 | 312.97 | 300.38 | 328.43 | 331.13 | 303.72 | 315.33 ± 12.53^{###} |
| Formula | 0.1 | | | 326.61 | 381.47 | | |
| II | | 346.49 | 347.86 | | | 353.63 | 351.21 ± 17.66^{###} |
| | 0.5 | 420.10 | 338.84 | 355 | 334.83 | 342.18 | 358.19 ± 31.68^{###} |
| | 1 | 365.93 | 372.44 | 366.76 | 392.9 | 373.9 | 374.39 ± 9.76^{###} |

***P < 0.01 compared with the control group;
^{###}P < 0.01 compared with the model group.

### Experimental Example 5: Detection Experiment of Compounds in the Example Using an Ischemic Heart Disease Mouse Model Induced by Isoproterenol (ISO)

### Experimental Method:

Male C57BL/6 mice were allowed to acclimate to the environment and then randomly divided into 9 groups, namely the blank control group, the ISO model group, the ISO+diltiazem 20 mg/kg positive control drug group, the ISO+Formula I (0.1, 0.5, 2 mg/kg) groups, and the ISO+Formula II (0.1, 0.5, 2 mg/kg) groups. Mice in the drug administration groups were intragastrically administered the corresponding doses of Formula I and Formula II compounds once a day at the same time. Mice in the positive drug diltiazem group were intragastrically administered the corresponding dose of diltiazem once a day. Mice in the blank control group and the model group were given the same amount of solvent (0.5% CMC-Na). The administration volume was 10 mL/kg, and the administration was continuous for 11 days.

From the 4th to the 10th day of drug administration, ISO modeling was carried out. The blank control group was subcutaneously injected with normal saline, and mice in the other groups were subcutaneously injected with ISO at a dose of 40 mg/kg (modeling was performed 2 hours after drug administration), for a total of 7 times.

Detection of Myocardial Hypertrophy/Heart Failure Biomarkers: After the drug administration ended, the animals' eyes were enucleated to collect blood. The blood was left to stand at room temperature for 2 hours, and then centrifuged at 3500 rpm for 20 minutes to separate the serum. The contents of serum ANP and NT-proBNP were determined according to the instructions of the ELISA kit.

Myocardial enzyme CK-MB detection: After the administration ended, blood was collected from the animals by enucleation. The blood was left to stand at room temperature for 2 hours, and then centrifuged at 3500 rpm for 20 minutes to separate the serum. The content of CK-MB in the serum was determined according to the instructions of the ELISA kit.

Calcium ion detection in myocardial tissue: After the experiment ended, some myocardial tissue was taken to prepare a 10% myocardial tissue homogenate with ultrapure water. The homogenate was centrifuged at 1200 rpm for 20 minutes at 4° C. to separate the supernatant of the myocardial tissue homogenate. The calcium ion content was determined according to the kit instructions; protein quantification was performed using the BCA protein assay method.

Pathological detection of myocardial tissue: After the experiment ended, the heart was washed with ice-cold physiological saline and dried with absorbent paper. It was then cut transversely into two halves. One-third of the part closer to the apex of the heart was fixed in a 4% paraformaldehyde solution. After dehydration with gradient ethanol and clearing with xylene, it was immersed in melted paraffin three times for embedding, 30 minutes each time. After embedding, continuous sections (about 4 μm thick) were made. Routine hematoxylin and eosin (H.E.) staining was performed. The pathological state of the myocardial tissue was observed under an optical microscope, photographed, and analyzed.

### Experimental Results:

Serum Biomarkers for Myocardial Hypertrophy/Heart Failure results: Serum N-terminal pro-B-type natriuretic peptide (NT-proBNP) is a biomarker for the early diagnosis of heart failure, and a decrease in its level is also strong evidence of the effectiveness of drug treatment. Atrial natriuretic peptide (ANP) is another sensitive serum biomarker for heart failure. Current research also shows that the levels of NT-proBNP and ANP are positively correlated with the degree of myocardial hypertrophy and can be used as biomarkers for myocardial hypertrophy. The levels of serum NT-proBNP and ANP were detected using ELISA kits, and the results are shown in Table 5. The levels of serum NT-proBNP and ANP in the model group were significantly higher than those in the blank control group, indicating severe heart damage in the mice, a possible heart failure, and the presence of myocardial hypertrophy symptoms. Doses of 0.1 mg/kg, 0.5 mg/kg, and 2 mg/kg of Formula II significantly reduced the levels of serum NT-proBNP and ANP. Doses of 0.1 mg/kg, 0.5 mg/kg, and 2 mg/kg of Formula I also significantly reduced the level of serum ANP. There were statistically significant differences compared with the model group. The three dose groups of Formula I and Formula II had a better effect on reducing ANP than the positive control drug diltiazem at 20 mg/kg. The three dose groups of Formula II also had a better effect on reducing NT-proBNP than the positive control drug diltiazem at 20 mg/kg.

TABLE 5
Effects of Formula I and Formula II on the Contents
of Serum Myocardial Hypertrophy/Heart Failure Biomarkers
NT-proBNP and ANP in Mice with Ischemic Heart Disease
Induced by Isoproterenol (ISO) (n = 8-9)

| Group | Dosage(mg/kg) | ANP(pg/mL) | NT-proBNP(pg/mL) |
|---|---|---|---|
| Control | — | 210.73 ± 10.22 | 34.93 ± 7.41 |
| Model | — | 421.27 ± 123.93*** | 46.19 ± 10.44* |
| dilti- | 20 | 361.59 ± 46.95 | 43.12 ± 2.93 |
| azem | | | |
| Formula | 0.1 | 301.02 ± 58.98^{#} | 39.35 ± 2.83 |
| I | 0.5 | 295.85 ± 57.72^{#} | 43.52 ± 6.06 |
| | 2 | 289.01 ± 50.17^{##} | 40.47 ± 7.15 |
| Formula | 0.1 | 303.60 ± 37.76^{#} | 34.07 ± 4.10^{##} |
| II | 0.5 | 264.38 ± 27.96^{##} | 31.31 ± 2.24^{###} |
| | 2 | 258.19 ± 52.28^{##} | 31.12 ± 2.33^{###} |

*P < 0.05, ***P < 0.001, compared with the control group;
^{#}P < 0.05, ^{##}P < 0.01, ^{###}P < 0.001, compared with the model group.

Myocardial injury serum biomarkers-Myocardial enzyme CK-MB results: Serum creatine kinase-isoenzyme (CK-MB) is another clinically specific and sensitive indicator for judging myocardial necrosis. The results are shown in Table 6. The level of serum myocardial enzyme CK-MB in the ISO model group of mice was significantly higher than that in the blank control group, further indicating the presence of heart injury. Formula I at 0.5 mg/kg, Formula II at 0.5 mg/kg and 2 mg/kg could significantly reduce the serum CK-MB level, showing a statistically significant difference compared with the model group. Formula I at 0.1 mg/kg and 2 mg/kg, and Formula II at 0.1 mg/kg also had a trend of reducing the serum CK-MB level. The effect of Formula I and Formula II in reducing CK-MB was better than that of the positive control drug diltiazem at 20 mg/kg.

TABLE 6
Effects of Formula I and Formula II on the content of
serum myocardial enzyme CK-MB in mice with isoproterenol
(ISO)-induced ischemic heart disease (n = 8-9)

| Group | Dosage (mg/kg) | CK-MB (ng/mL) |
|---|---|---|
| Control | — | 12.73 ± 0.85 |
| Model | — | 18.75 ± 5.50* |
| diltiazem | 20 | 17.98 ± 2.88 |
| Formula I | 0.1 | 14.86 ± 2.90 |
| | 0.5 | 13.71 ± 2.75^{#} |
| | 2 | 15.05 ± 2.49 |
| Formula II | 0.1 | 15.48 ± 1.76 |
| | 0.5 | 12.89 ± 2.29^{##} |
| | 2 | 12.91 ± 1.53^{##} |

*P < 0.05, compared with the control group;
^{#}P < 0.05, ^{##}P < 0.01, compared with the model group.

Results of calcium ion concentration in myocardial tissue: Calcium ions (Ca²⁺) in myocardial tissue play an important role in the excitation-contraction coupling of myocardial cells. Calcium overload is one of the important mechanisms in isoproterenol (ISO)-induced myocardial hypertrophy injury. The results are shown in Table 7. The Ca²⁺ content in the myocardial tissue of mice in the ISO model group was significantly higher than that in the blank control group, indicating obvious calcium overload in the myocardial tissue. All three dose groups of Formula I and Formula II could significantly reduce the Ca²⁺ content in the myocardial tissue. The positive control drug diltiazem at a dose of 20 mg/kg could also significantly reduce the Ca²⁺ content in the myocardial tissue. The low and medium dose groups of Formula I and Formula II had a better effect on improving the calcium overload phenomenon in the myocardial tissue than the positive control drug diltiazem at a dose of 20 mg/kg.

TABLE 7
Effects of Formula I and Formula II on the Ca²⁺ content
in the myocardial tissue of mice with myocardial ischemia
induced by isoproterenol (ISO) (n = 8-9)

| Group | Dosage (mg/kg) | Ca²⁺ (mmol/gprot) |
|---|---|---|
| Control | — | 0.041 ± 0.017 |
| Model | — | 0.076 ± 0.011*** |
| diltiazem | 20 | 0.057 ± 0.011^{##} |
| Formula I | 0.1 | 0.050 ± 0.008^{###} |
| | 0.5 | 0.046 ± 0.010^{###} |
| | 2 | 0.058 ± 0.009^{##} |
| Formula II | 0.1 | 0.046 ± 0.015^{###} |
| | 0.5 | 0.054 ± 0.014^{##} |
| | 2 | 0.059 ± 0.009^{##} |

***P < 0.001, compared with the control group;
^{##}P < 0.01, ^{###}P < 0.001, compared with the model group.

Results of myocardial tissue pathological examination: The results of H&E staining of myocardial tissue are shown in FIG. 2. In the blank control group, the myocardial tissue fibers of mice were arranged neatly, with clear structures showing regular patterns. The cell nuclei were clear, the cytoplasm was rich and uniform, and there was little infiltration of inflammatory cells. In the ISO model group, the myocardial cells of the ventricular septum and papillary muscles of mice were significantly hypertrophied, with myocardial cell edema and degeneration. The local ventricular septum and papillary muscle myocardial tissue were severely damaged (myocardial cell necrosis). The damaged (necrotic) lesions were distributed in patches and connected to each other. Transmural myocardial tissue damage of the ventricular septum and papillary muscles was locally visible. Inflammation was significant in the lesion sites, and obvious fibrous tissue hyperplasia was observed in the damaged areas. Administration of three doses of Formula I and Formula II by gavage could significantly improve the pathological damage of myocardial tissue in mice induced by ISO. The myocardial cell edema and hypertrophy were alleviated, the degree of myocardial cell necrosis was reduced, and the degree of fibrous tissue hyperplasia was decreased. The pathological scores of heart tissues are shown in Table 8. Three doses of Formula I and Formula II significantly reduced the number of animals with severe myocardial tissue lesions and the average lesion degree score. The positive control drug diltiazem at a dose of 20 mg/kg could also significantly reduce the degree of myocardial tissue lesions. The improvement effects of Formula I at 2 mg/kg and Formula II at 2 mg/kg on the degree of myocardial tissue lesions were better than those of the positive control drug diltiazem at 20 mg/kg.

TABLE 8
Effects of Formula I and Formula II on the degree of myocardial tissue lesions in
mice with isoproterenol (ISO)-induced myocardial ischemia (H&E 200×)

| | | |
|---|---|---|
| | The number of animals with different | |
| | degrees of lesions | |
| Dosage | Grading of lesion degree | average |

| Group | (mg/kg) | − | + | ++ | +++ | ++++ | +++++ | score |
|---|---|---|---|---|---|---|---|---|
| Control | — | 10 | 0 | 0 | 0 | 0 | 0 | 0.00 ± 0.00 |
| Model | — | 0 | 0 | 0 | 0 | 4 | 6 | 4.60 ± 0.52*** |
| diltiazem | 20 | 2 | 3 | 1 | 4 | 0 | 0 | 1.70 ± 1.25^{###} |
| Formula I | 0.1 | 0 | 0 | 2 | 5 | 2 | 0 | 3.00 ± 0.71^{###} |
| | 0.5 | 0 | 2 | 4 | 4 | 0 | 0 | 2.20 ± 0.79^{###} |
| | 2 | 1 | 6 | 3 | 0 | 0 | 0 | 1.20 ± 0.63^{###} |
| Formula II | 0.1 | 0 | 2 | 4 | 4 | 0 | 0 | 2.20 ± 0.79^{###} |
| | 0.5 | 0 | 3 | 4 | 3 | 0 | 0 | 2.00 ± 0.82^{###} |
| | 2 | 4 | 6 | 1 | 0 | 0 | 0 | 0.73 ± 0.65^{###} |

***P < 0.001, compared with the control group;
^{###}P < 0.001, compared with the model group.

### APPENDIX: PATHOLOGICAL SCORING CRITERIA

−: No abnormalities were observed.
+: No myocardial tissue damage was seen. There was local hypertrophy of cardiomyocytes in the interventricular septum and papillary muscles, local edema and degeneration of cardiomyocytes, widening of the local muscle space in the lesion area, and mild non-specific inflammation in the interstitium.
++: No obvious myocardial tissue damage (cardiomyocyte necrosis) was observed. There was local hypertrophy of cardiomyocytes in the interventricular septum and papillary muscles, local edema and degeneration of cardiomyocytes. Myofibril dissolution (disappearance of cross-striations) could be seen in local areas. The lesions were relatively limited and distributed in small foci. Non-specific inflammation was visible in the interstitium, and local fibrous tissue hyperplasia in the interstitium was noted.
+++: There was obvious hypertrophy of cardiomyocytes in the interventricular septum and papillary muscles, local edema and degeneration of cardiomyocytes. There was damage to the myocardial tissue of the local interventricular septum or papillary muscles (cardiomyocyte necrosis, only one site was observed in the sample). The lesion was limited to the subendocardium of the local interventricular septum (or papillary muscle), with a limited and small affected area. Non-specific inflammation was visible in the interstitium of the lesion area, and local fibrous tissue hyperplasia in the interstitium was noted.
++++: The hypertrophy of cardiomyocytes in the interventricular septum and papillary muscles is remarkable. Locally, there is edema and degeneration of cardiomyocytes; there is obvious damage (necrosis of cardiomyocytes) in the myocardial tissue of the local interventricular septum and papillary muscles. The lesions are distributed in foci (or small patches), and the affected area is relatively large. In the lesion sites, inflammation is obvious, and local fibrous tissue proliferates significantly.
+++++: The hypertrophy of cardiomyocytes in the interventricular septum and papillary muscles is remarkable. Locally, there is edema and degeneration of cardiomyocytes; the damage (necrosis of cardiomyocytes) of the myocardial tissue of the local interventricular septum and papillary muscles is severe. The damage (necrosis) lesions are distributed in patches and are connected to each other. Transmural myocardial tissue damage of the interventricular septum and papillary muscles can be seen locally. In the lesion sites, inflammation is prominent, and obvious fibrous tissue proliferation can be seen in the damaged areas.

### Experimental Example 6: Detection Experiment of the Compound in Example Using a Mouse Model of Hypertensive Heart Disease Induced by Angiotensin II (AngII)

### Experimental Methods:

After male C57BL/6 mice were acclimated to the environment, they were randomly divided into 10 groups, namely the blank control group, the AngII model group, the AngII+telmisartan 10 mg/kg positive control group, the AngII+spironolactone 20 mg/kg positive control group, the AngII+Formula I (0.1, 0.5, 2 mg/kg) groups, and the AngII+Formula II (0.1, 0.5, 2 mg/kg) groups. The mice in the drug administration groups were intragastrically administered the corresponding doses of Formula I and Formula II compounds once a day at the same time. The mice in the positive control groups were intragastrically administered the corresponding doses of telmisartan and spironolactone once a day. The mice in the blank control group and the model group were given the same amount of solvent (0.5% CMC-Na solution). The administration volume was 10 mL/kg, and the administration was continuous for 14 days.

From the 1st to the 13th day of drug administration, AngII modeling was carried out. The mice in the blank control group were subcutaneously injected with normal saline, and the mice in the other groups were subcutaneously injected with 1.44 mg/kg of AngII (modeling was carried out 2 h after drug administration), for a total of 13 times.

Blood pressure monitoring: The blood pressure of mice was measured on the 0th, 7th, and 13th days of drug administration. Three mice were randomly selected from each group. The mice were placed in a fixator to expose the tail. The fixator was placed on a heating pad to maintain the normal body temperature of the mice. The root of the tail was passed through a pressurized tail cuff and fixed. Inflation and pressurization were started, and a measuring instrument was used to measure the systolic blood pressure (SBP). The blood pressure index of each animal was continuously measured 5 times, and the average value was taken.

Echocardiogram detection: On the 14th day of drug administration (after 13 times of AngII modeling), 5 animals were randomly selected from each group. After anesthesia and fixation, the chest hair was shaved, and a small amount of coupling agent was applied to the chest. The Vevo 770 Imaging System, a small animal ultrasonic real-time imaging system, was used for echocardiogram examination. All echocardiogram indexes of the animals were continuously measured for 3 cardiac cycles, and the average value was taken.

Detection of myocardial hypertrophy/heart failure biomarkers: After the experiment, the animals were sacrificed by enucleation to obtain blood. The blood was left to stand at room temperature for 2 hours, and then centrifuged at 3500 rpm for 20 minutes to separate the serum. The content of serum BNP was determined according to the instructions of the ELISA kit.

Detection of serum biomarkers of myocardial injury: After the experiment, the animals were sacrificed by enucleation to obtain blood. The blood was left to stand at room temperature for 2 hours, and then centrifuged at 3500 rpm for 20 minutes to separate the serum. The contents of serum cardiac troponin cTn-T and myocardial enzyme CK-MB were determined according to the instructions of the ELISA kit.

Detection of serum inflammatory factors: After the experiment, the animals were sacrificed by enucleation to obtain blood. The blood was left to stand at room temperature for 2 hours, and then centrifuged at 3500 rpm for 20 minutes to separate the serum. The content of serum TNF-α was determined according to the instructions of the ELISA kit.

Detection of serum aldosterone (ALD) level: After the experiment, the animals were sacrificed by enucleation to obtain blood. The blood was left to stand at room temperature for 2 hours, and then centrifuged at 3500 rpm for 20 minutes to separate the serum. The content of serum ALD was determined according to the instructions of the ELISA kit.

Detection of serum CAMP level: After the experiment, the animals were sacrificed by enucleation to obtain blood. The blood was left to stand at room temperature for 2 hours, and then centrifuged at 3500 rpm for 20 minutes to separate the serum. The content of serum cAMP was determined according to the instructions of the ELISA kit.

### Experimental Results:

Blood pressure monitoring results: The results are shown in Table 9. Before drug administration, there was no significant difference in the basic systolic blood pressure (SBP value) of mice in each group. After continuous stimulation with angiotensin II (AngII) for 7 days and 14 days, the SBP of mice in the model group increased significantly, showing a statistically significant difference compared with the blank control group. The three dose groups of Formula I and Formula II significantly reduced the SBP value, and there were statistically significant differences compared with the model group. The positive control drugs spironolactone and telmisartan could also significantly reduce the SBP value. The activity of reducing SBP in each dose group of Formula I and Formula II was comparable to that of spironolactone at a dose of 20 mg/kg. After administration of telmisartan at a dose of 10 mg/kg, the SBP value of mice was lower than that of the animals in the blank control group. Among them, the SBP value on the 7th day was significantly lower than that of the blank control group, showing a state of hypotension, which was consistent with the existing clinical reports. Taking ARB drugs such as telmisartan may induce the side effect of transient hypotension, and no such adverse effects were observed in Formula I and Formula II.

TABLE 9
Effects of Formula I and Formula II administration on SBP of mice with
hypertensive heart disease induced by angiotensin II (AngII) (n = 3)

| | Dosage | | | |
|---|---|---|---|---|
| Group | (mg/kg) | 0 d | 7 d | 14 d |
| Control | — | 104.13 ± 9.96 | 106.13 ± 4.74 | 103.53 ± 6.99 |
| Model | — | 107.47 ± 3.93 | 134.87 ± 4.39** | 144.80 ± 5.05** |
| Spironolactone | 20 | 106.13 ± 0.81 | 109.73 ± 6.81^{##} | 109.70 ± 4.20^{###} |
| Telmisartan | 10 | 104.13 ± 3.56 | 91.20 ± 6.35^{###&} | 100.47 ± 7.68^{##} |
| Formula I | 0.1 | 102.13 ± 4.30 | 121.33 ± 3.19^{#} | 107.60 ± 9.81^{##} |
| | 0.5 | 109.70 ± 5.67 | 109.80 ± 4.50^{##} | 105.13 ± 7.10^{##} |
| | 2 | 104.93 ± 9.21 | 107.73 ± 6.53^{##} | 109.20 ± 5.19^{##} |
| Formula II | 0.1 | 100.30 ± 7.37 | 105.73 ± 6.00^{##} | 110.13 ± 3.51^{###} |
| | 0.5 | 102.43 ± 8.41 | 106.07 ± 1.22^{###} | 113.93 ± 3.51^{###} |
| | 2 | 101.27 ± 12.13 | 110.93 ± 5.78^{##} | 108.27 ± 7.29^{##} |

**P < 0.01, compared with the control group;
^{#}P < 0.05, ^{##}P < 0.01, ^{###}P < 0.001, compared with the model group;
^{&}P < 0.05, compared with the control group.

Results of echocardiography examination: Small animal echocardiography technology is a simple and highly repeatable technique for evaluating the cardiac function of mice. The M-mode echocardiogram was obtained using this technology (see FIG. 4). The data were measured and calculated, and some of the data are shown in Table 10. Continuous administration of AngII led to significant left ventricular dysfunction, causing a significant increase in the left ventricular wall thickness during systole and diastole, presenting obvious signs of myocardial hypertrophy. The three dose groups of Formula I and Formula II were able to significantly reduce the posterior wall thickness of the left ventricle during systole and diastole. There were statistically significant differences compared with the model group, and significantly improved myocardial hypertrophy. The positive control drugs, spironolactone and telmisartan, could also significantly reduce the posterior wall thickness of the left ventricle during systole and diastole. The improvement activity of the three dose groups of Formula I and Formula II on myocardial hypertrophy was better than that of spironolactone at a dose of 20 mg/kg.

TABLE 10
Effects of Formula I and Formula II administration
on echocardiogram data of hypertensive heart disease
mice induced by angiotensin II (AngII) (n = 5)

| | | Posterior wall | Post-diastolic |
|---|---|---|---|
| | Dosage | thickness in | posterio rwall |
| Group | (mg/kg) | systole (mm) | thickness (mm) |
| Control | — | 0.93 ± 0.08 | 0.72 ± 0.03 |
| Model | — | 1.35 ± 0.12*** | 1.07 ± 0.19** |
| Spironolactone | 20 | 1.01 ± 0.08^{###} | 0.81 ± 0.07^{#} |
| Telmisartan | 10 | 0.79 ± 0.16^{###} | 0.65 ± 0.07^{##} |
| Formula I | 0.1 | 0.90 ± 0.06^{###} | 0.79 ± 0.09^{#} |
| | 0.5 | 0.87 ± 0.11^{###} | 0.64 ± 0.02^{###} |
| | 2 | 0.90 ± 0.16^{##} | 0.67 ± 0.08^{##} |
| Formula II | 0.1 | 0.91 ± 0.10^{###} | 0.72 ± 0.08^{##} |
| | 0.5 | 0.87 ± 0.04^{###} | 0.69 ± 0.06^{##} |
| | 2 | 0.82 ± 0.11^{###} | 0.72 ± 0.06^{##} |

**P < 0.01, ***P < 0.001, compared with the control group;
^{#}P < 0.05, ^{##}P < 0.01, ^{###}P < 0.001, compared with the model group.

Results of biomarkers for myocardial hypertrophy/heart failure: Serum brain natriuretic peptide (BNP) is another important biomarker for the early diagnosis of heart failure. Meanwhile, it is positively correlated with the degree of myocardial hypertrophy and is one of the biomarkers for myocardial hypertrophy. The results are shown in Table 11. The serum BNP level in the AngII model group of mice was significantly higher than that in the blank control group. All three doses of Formula I and Formula II significantly reduced the serum BNP level, showing statistically significant differences compared with the model group. The positive control drugs, spironolactone and telmisartan, could also significantly reduce the serum BNP level. The three dose groups of Formula I and Formula II had a better effect in reducing BNP than the positive control drug spironolactone at a dose of 20 mg/kg.

TABLE 11
Effects of administration of Formula I and Formula II
on the content of serum biomarker BNP for myocardial
hypertrophy/heart failure in mice with hypertensive heart
disease induced by angiotensin II (AngII) (n = 7-9)

| Group | Dosage (mg/kg) | BNP (pg/mL) |
|---|---|---|
| Control | — | 87.43 ± 8.13 |
| Model | — | 146.74 ± 10.80*** |
| Spironolactone | 20 | 129.92 ± 15.60^{#} |
| Telmisartan | 10 | 96.84 ± 7.34^{###} |
| Formula I | 0.1 | 112.93 ± 17.57^{###} |
| | 0.5 | 117.59 ± 13.78^{###} |
| | 2 | 102.97 ± 5.56^{###} |
| Formula II | 0.1 | 103.21 ± 16.09^{###} |
| | 0.5 | 101.08 ± 9.09^{###} |
| | 2 | 91.67 ± 15.39^{###} |

***P < 0.001, compared with the control group;
^{#}P < 0.05, ^{###}P < 0.001, compared with the model group.

Results of serum biomarkers of myocardial injury: When myocardial cells necrose, some protein substances contained in the myocardium are released from the myocardial tissue and appear in the peripheral circulating blood, which can be used as judgment indicators of myocardial injury. Troponin (Tn) is a regulatory protein for myocardial tissue contraction. Cardiac troponin (cTn) is different from Tn in skeletal muscle in terms of molecular structure and immunology. Therefore, it is unique to the myocardium and is the most specific and sensitive first-choice marker for judging myocardial necrosis. cTn has three subtypes: cTn-T, cTn-I, and cTn-C. cTn-T has high diagnostic value for both early and late myocardial necrosis. As shown in Table 12, the serum level of the myocardial injury marker protein cTn-T in the model group of mice was significantly higher than that in the blank control group. Administration of three doses of Formula I and Formula II could significantly reduce the serum cTn-T level, showing a statistically significant difference compared with the model group. The positive control drugs spironolactone and telmisartan could also significantly reduce the serum cTn-T level. The reducing effect of Formula I on cIn-T was comparable to that of spironolactone at a dose of 20 mg/kg, and the reducing effect of Formula II on cTn-T was better than that of spironolactone at a dose of 20 mg/kg.

Serum creatine kinase isoenzyme (CK-MB) is another clinically specific and sensitive indicator for judging myocardial necrosis. The results are shown in Table 12. The level of serum myocardial enzyme CK-MB in the AngII model group of mice was significantly higher than that in the blank control group. Administration of three doses of Formula I and Formula II could significantly reduce the serum CK-MB level. The positive control drug telmisartan could also significantly reduce the serum CK-MB level. The effect of Formula I and Formula II in reducing CK-MB was better than that of the positive control drug spironolactone at a dose of 20 mg/kg.

TABLE 12
Effects of administration of Formula I and Formula
II on the contents of serum myocardial injury biomarkers
cTn-T and CK-MB in hypertensive heart disease mice
induced by angiotensin II (AngII) (n = 7-9)

| | Dosage | | |
|---|---|---|---|
| Group | (mg/kg) | cTn-T (ng/L) | CK-MB (ng/mL) |
| Control | — | 122.68 ± 9.84 | 12.83 ± 1.87 |
| Model | — | 222.14 ± 49.60*** | 20.14 ± 2.08*** |
| Spironolactone | 20 | 169.17 ± 21.95^{#} | 18.86 ± 1.40 |
| Telmisartan | 10 | 134.07 ± 17.50^{###} | 12.72 ± 1.09^{###} |
| Formula I | 0.1 | 167.25 ± 24.56^{#} | 15.73 ± 1.79^{###} |
| | 0.5 | 165.79 ± 23.78^{##} | 15.17 ± 1.63^{###} |
| | 2 | 164.52 ± 19.68^{##} | 15.72 ± 1.89^{###} |
| Formula II | 0.1 | 153.56 ± 13.05^{##} | 15.36 ± 2.41^{###} |
| | 0.5 | 145.15 ± 15.79^{###} | 14.07 ± 1.33^{###} |
| | 2 | 136.64 ± 18.24^{###} | 13.39 ± 2.57^{###} |

***P < 0.001, compared with the control group;
^{#}P < 0.05, ^{###}P < 0.001, compared with the model group.

Results of serum inflammatory factors: The generation of inflammation is a key factor in the occurrence of cardiovascular diseases. The release of inflammatory factors (such as TNF-α) will directly induce cardiomyocyte apoptosis and impairment of cardiac contractility, and even lead to heart failure. The results are shown in Table 13. The level of serum inflammatory factor TNF-α in the model group of mice was significantly higher than that in the blank control group, indicating an obvious inflammatory response in the body. Administration of three doses of Formula I and Formula II could significantly reduce the serum TNF-α level. The positive control drugs, spironolactone and telmisartan, could also significantly reduce the serum TNF-α level. The reducing effect of the three dose groups of Formula I and Formula II on TNF-α was better than that of the positive control drug spironolactone at a dose of 20 mg/kg.

TABLE 13
Effects of Formula I and Formula II administration on the content
of serum inflammatory factor TNF-α in mice with hypertensive
heart disease induced by angiotensin II (Ang II) (n = 7-9)

| Group | Dosage (mg/kg) | TNF-α (ng/L) |
|---|---|---|
| Control | — | 571.91 ± 60.44 |
| Model | — | 850.37 ± 84.90*** |
| Spironolactone | 20 | 758.49 ± 72.35^{#} |
| Telmisartan | 10 | 616.87 ± 70.42^{###} |
| Formula I | 0.1 | 706.64 ± 91.15^{##} |
| | 0.5 | 699.02 ± 55.58^{###} |
| | 2 | 634.24 ± 98.58^{###} |
| Formula II | 0.1 | 645.36 ± 87.50^{###} |
| | 0.5 | 671.09 ± 80.55^{###} |
| | 2 | 627.56 ± 98.89^{###} |

***P < 0.001, compared with the control group;
^{#}P < 0.05, ^{##}P < 0.01, ^{###}P < 0.001, compared with the model group.

AngII stimulates downstream signaling pathways by binding to the cardiac angiotensin receptor 1 (AT1R), leading to an increase in aldosterone (ALD) production. In addition to regulating sodium reabsorption and blood volume, ALD has a direct effect on promoting cardiac hypertrophy. Moreover, ALD can further enhance the sensitivity of myocardial cells to AngII by increasing the density of cardiac AT1R. The results are shown in Table 14. The serum ALD level in the model group was significantly higher than that in the blank control group. Administration of Formula I and Formula II at all doses significantly reduced the serum ALD levels. The positive control drugs spironolactone and losartan also significantly reduced the serum ALD levels. The reduction in ALD by the three doses of Formula I and Formula II was better than that of the positive control drug spironolactone at 20 mg/kg.

TABLE 14
Effects of Formula I and Formula II administration on
the serum ALD content in mice with hypertensive heart
disease induced by angiotensin II (AngII) (n = 7-9)

| | Dosage | |
|---|---|---|
| Group | (mg/kg) | ALD (pg/mL) |
| Control | — | 158.79 ± 9.20 |
| Model | — | 244.19 ± 30.00*** |
| Spironolactone | 20 | 209.03 ± 25.11^{#} |
| Telmisartan | 10 | 162.74 ± 7.38^{###} |
| Formula I | 0.1 | 206.80 ± 16.10^{##} |
| | 0.5 | 192.69 ± 14.38^{###} |
| | 2 | 199.41 ± 31.27^{#} |
| Formula II | 0.1 | 186.44 ± 21.59^{###} |
| | 0.5 | 177.28 ± 21.57^{###} |
| | 2 | 185.89 ± 12.61^{###} |

***P < 0.001, compared with the control group;
^{#}P < 0.05, ^{##}P < 0.01, ^{###}P < 0.001, compared with the model group.

Results of the sympathetic nervous system endocrine factor cyclic adenosine monophosphate (cAMP): Overexcitation of the sympathetic nervous system can lead to an excessive increase in the content of cyclic adenosine monophosphate (cAMP), induce arrhythmia or activate the signal transduction pathway of cardiomyocytes, resulting in myocardial hypertrophy, fibrosis, and even affecting cardiac function. The results are shown in Table 15. The serum cAMP level in the model group of mice was significantly higher than that in the blank control group, indicating an excessive increase in the excitability of the sympathetic nervous system. Formula I at 0.1 mg/kg and 0.5 mg/kg, and Formula II at 0.1 mg/kg, 0.5 mg/kg, and 2 mg/kg could all significantly reduce the serum cAMP level. The positive control drug telmisartan could also significantly reduce the serum cAMP level, while spironolactone had no significant effect on reducing the excessively increased CAMP level. The reducing effect of the three dose groups of Formula II on the excessively increased cAMP was comparable to that of the positive control drug telmisartan at 10 mg/kg.

TABLE 15
Effects of Formula I and Formula II administration on
the serum cAMP content in mice with hypertensive heart
disease induced by angiotensin II (Ang II) (n = 7-9)

| Group | Dosage (mg/kg) | cAMP (nmol/L) |
|---|---|---|
| Control | — | 25.92 ± 2.09 |
| Model | — | 31.21 ± 1.65*** |
| Spironolactone | 20 | 29.34 ± 3.04 |
| Telmisartan | 10 | 25.18 ± 1.10^{###} |
| Formula I | 0.1 | 27.73 ± 3.03^{#} |
| | 0.5 | 27.42 ± 1.12^{###} |
| | 2 | 29.04 ± 5.17 |
| Formula II | 0.1 | 24.81 ± 1.63^{###} |
| | 0.5 | 26.38 ± 1.81^{###} |
| | 2 | 24.42 ± 2.07^{###} |

***P < 0.001, compared with the control group;
^{#}P < 0.05, ^{###}P < 0.001, compared with the model group.

1. A methyl ophiopogonone A derivative represented by the following compound I, II and III or the pharmaceutically acceptable salt thereof:

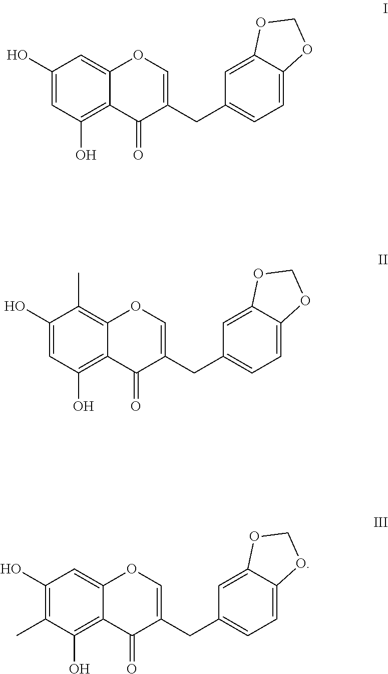 [SMILES: O=c1c(Cc2ccc3c(c2)OCO3)coc2cc(O)cc(O)c12 ; Cc1c(O)cc(O)c2c(=O)c(Cc3ccc4c(c3)OCO4)coc12 ; Cc1c(O)cc2occ(Cc3ccc4c(c3)OCO4)c(=O)c2c1O]

2. A method for preparing compound I of methyl ophiopogonone A derivative according to claim 1, characterized by comprising the following steps:
under basic conditions, reacting 2,4,6-trihydroxyacetophenone (1) with a halo(methoxy)methyl ether to produce a compound of Formula (2);
reacting the compound of Formula (2) with piperonal under basic conditions to produce a compound of Formula (3);
reducing the double bond in the compound of Formula (3) to produce a compound of Formula (4);
reacting the compound of Formula (4) with phosphorus oxychloride and N, N-dimethylformamide to produce compound I:

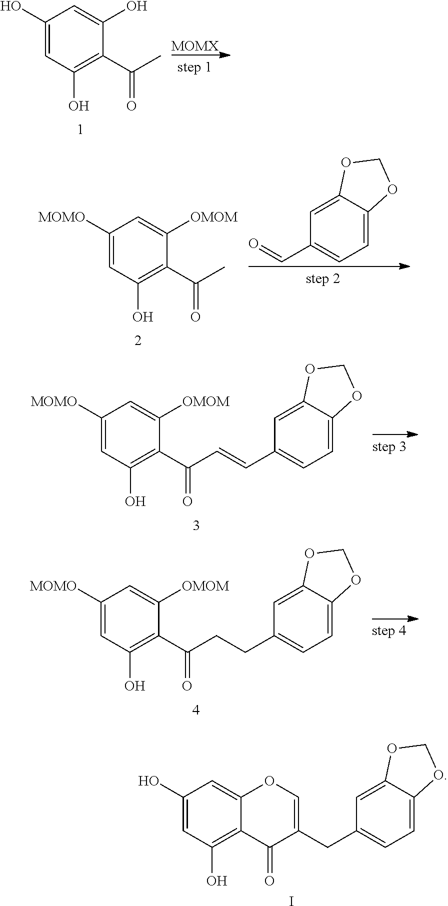

3. The method for preparing compound I of methyl ophiopogonone A derivative according to claim 2, characterized in that: in the first step, the base is triethylamine or N, N-diisopropylethylamine, and the halomethoxymethyl ether is bromomethoxymethyl ether; in the second step, the base is sodium hydride or sodium alkoxide, KOH/EtOH; in the third step, the reducing agent is palladium on carbon/hydrogen or stannous chloride.

4. A method for preparing compounds II and III of methyl ophiopogonone A derivative according to claim 1, characterized by comprising the following steps:
reducing 2, 4, 6-trihydroxybenzaldehyde to 2,4,6-trihydroxytoluene, and then reacting 2,4,6-trihydroxytoluene with acetic anhydride to produce a compound of Formula (5);
under basic conditions, reacting the compound of Formula (5) with a halomethoxymethyl ether to produce a compound of Formula (6);
under basic conditions, reacting the compound of Formula (6) with piperonal to produce a compound of Formula (7);
reducing the double bond in the compound of Formula (7) to produce a compound of Formula (8);
reacting the compound of Formula (8) with phosphorus oxychloride and N, N-dimethylformamide to produce compounds II and III:

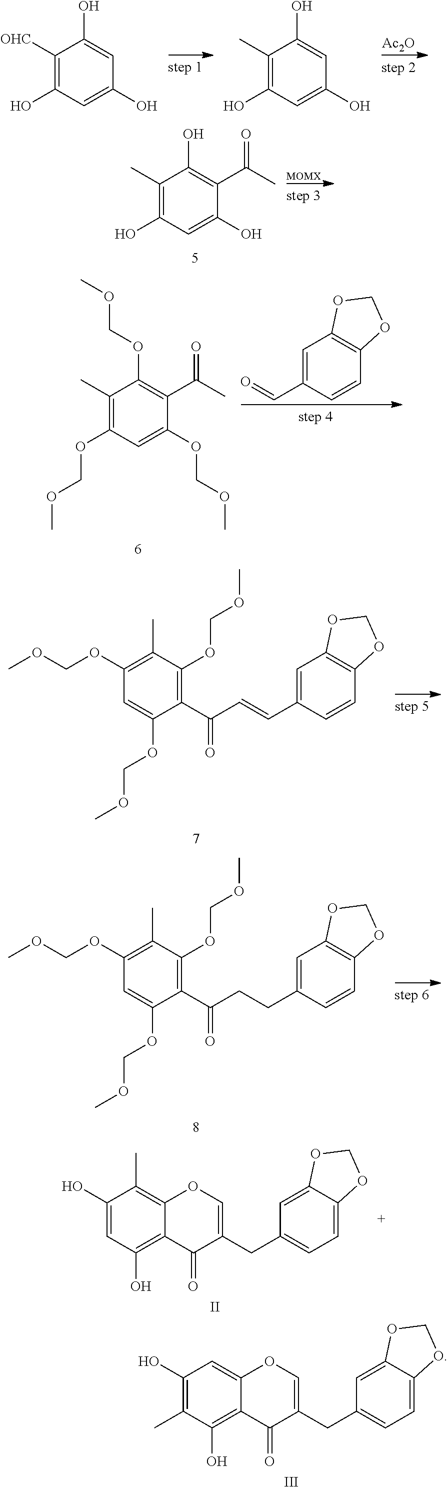

5. The method for preparing compounds II and III of methyl ophiopogonone A derivative according to claim 4, characterized in that: in the first step, the reducing agent is a metal reducing agent or a metal borohydride; in the third step, the base is triethylamine or N, N-diisopropylethylamine, and the halomethoxymethyl ether is bromomethoxymethyl ether; in the fourth step, the base is sodium hydride or sodium alkoxide, KOH/EtOH; in the fifth step, the reducing agent is palladium on carbon/hydrogen or stannous chloride.

6. A pharmaceutical composition, characterized by comprising a prophylactically and/or therapeutically effective amount of the methyl ophiopogonone A derivative or the pharmaceutically acceptable salt thereof according to claim 1, and optionally one or more pharmaceutically acceptable carriers or excipients.

7. Use of the methyl ophiopogonone A derivative or the pharmaceutically acceptable salt thereof according to claim 1, or the pharmaceutical composition according to claim 6, in the manufacture of a medicament for preventing and/or treating a cardiovascular diseases.

8. The use according to claim 7, characterized in that the cardiovascular disease is selected from the group consisting of: myocardial ischemia, myocardial injury, myocardial hypertrophy, coronary heart disease, hypertension, heart failure, cardiomyopathy, arrhythmia, myocardial infarction, and angina pectoris.
